(19) United States
(12) Patent Application Publication
(10) Pub. No.: US 2014/0302185 A1
(43) Pub. Date: Oct. 9, 2014

(54) COMPOSITION FOR THE TREATMENT OF SKIN LESIONS

(71) Applicant: FREIA FARMACEUTICI S.R.L., Milano (IT)

(72) Inventor: Alessandro Guido Cavalieri Manasse, Copparo (IT)

(73) Assignee: FREIA FARMACEUTICI S.R.L., Milano (IT)

(21) Appl. No.: 14/350,133

(22) PCT Filed: Oct. 4, 2012
(86) PCT No.: PCT/IB2012/055330

§ 371 Date: Apr. 7, 2014

(30) Foreign Application Priority Data
Oct. 5, 2011 (IT) MI2011A001806

(51) Int. Cl. A61K 36/185; A61K 31/728

(52) U.S. Cl. CPC A61K 36/185; A61K 31/728
USPC 424/774

(57) ABSTRACT

A composition is hereby described, comprising virgin oil of Cannabis sativa or a derivative thereof, hyaluronic acid, a salt or derivative thereof, and cosmetically or pharmaceutically acceptable excipients. Specifically, this composition has proved to be particularly suitable for the treatment of the skin both when affected by lesions, by intervening actively in the process of healing, and when subjected to treatment with radiations, by intervening actively in the prevention and reduction in the incidence of radio-dermatiti and dermatitis induced from the radiations.

### FIELD OF THE INVENTION

The present invention relates to a composition comprising virgin oil of Cannabis Sativa or a derivative thereof, hyaluronic acid, a salt or derivative thereof, and cosmetically or pharmaceutically acceptable excipients.

Specifically, this composition has proved to be particularly suitable for the treatment of the skin both when affected by lesions, by actively intervening in the process of healing, and when subjected to treatment with radiations, by actively intervening in the prevention and reduction in the incidence of radio-dermatitis and dermatitis induced by the radiations.

### STATE OF THE ART

Skin is not a simple protective covering, but an actual organ, composed of epidermis (epithelial tissue), dermis (connective tissue), and hypodermis (adipose tissue).

The epidermis acts as a barrier and is subdivided, from the deepest layer to the uppermost layer, in different layers according to the morphological characteristics that keratinocytes assume during the differentiation process: basal layer (or germinal), stratum spinosum (or polished), stratum granulosum and stratum corneum. The stratum corneum, the outermost, is made up of keratinised dead cells (keratin) that are continuously renewed and eliminated according to a cycle of 3-4 weeks.

The dermis is a connective tissue type, underlying the epidermis, characterised mainly by elastin fibres, which provide the correct elasticity to the skin, by collagen fibres, which support and resistance function and from the fundamental substance, which has a cementing function. Since it is rich in blood and lymph vessels, the skin has also nutrition function. In the dermis, there are several skin appendages, such as sudoriparous glands, piliferous follicles, and sebaceous glands.

The hypodermis is the third and deepest layer of the skin, directly in contact with the dermis on the one hand and with the subcutaneous fatty and muscle tissues, on the other hand. The hypodermis is made up of connective tissue particularly rich in adipocytes, the cells responsible for the biosynthesis of fats. Thanks to the presence of this cell type, this tissue acts as an energy reserve and, at the same time, as a thermal insulator and as a buffer.

In case of a wound, the skin starts a natural biological phenomenon: healing.

Healing is a complex process of repair during which the body stops bleeding, heals, and closes the wound. The injured tissue is then reconstructed and the damage is repaired.

From a physiological point of view, the healing process starts a few hours from the injury event and has the purpose of replacing the clot with a solid and definitive structure. It is featured by the cell proliferation of epithelial, endothelial, and connective tissue structures at the edges of the wound, which gives rise to a tissue called of granulation for its characteristic granulose appearance. At the edges of the wound, from the endothelium, starts the production of forged cells that, by following the scaffolding formed by the fibrin network, are lead towards the central area where they are welded with those coming from the opposite side. 24-72 hours after the trauma, there is an important proliferation of fibroblasts, cell elements that have the property of secreting hyaluronic acid. This substance is an active component in the formation of collagen fibres, sturdy structures that gradually will replace the fibrin strands. Fibroblasts, already at the end of the first week, are almost all the cells present in the wound; their activity will last up to the time necessary for the collagen product to fill the wound.

Simultaneously with the just described phase, begins also the cell proliferation of the epithelium basal layer. This tissue has the important function of covering the wound, but in the processes of healing by second intention, where the granulation tissue fills the wound from underneath and does not offer the adequate support to the progression of these cells, the mechanism is ineffective and determines hypertrophies and scar flaws.

The onset of a pathological healing process is therefore due to a qualitative or quantitative error of the connective tissue response that, in deficit, will give rise to atrophy; in excess to a hypertrophy of the scar.

When a scar does not undergo spontaneous regression after two months, but it is protruding, strongly hyperemic, hard, and painful, it is defined as hypertrophic. In particular, the hypertrophic scars can be irritating, unaesthetic and disfiguring the appearance of the person or, even more, limiting movements if they are located on or near the joints.

Instead, keloid is the most striking pathological healing type: just as the hypertrophic scar, it has a bright red colour, but is harder, up to having the appearance of a plastron with dilated capillaries on the surface thereof; a substantial difference with the latter, instead, is the extension of the keloid beyond the limits of the initial lesion, up to the point of creating real figured lesions in the human body.

The therapeutic approach aimed to ensure the proper healing process without the appearance of hypertrophic scars and by preventing the formation of keloids, consists basically in the removal of the hyperkeratotic areas and can follow the surgical/ablative path (cryotherapy, chemical peeling, curettage, dermabrasion, laser) or the pharmacological path. In the state of the art, the most covered pharmacological solutions involve the use of:
intralesional injections with steroidal preparations, the use of which is to be preferred in case of serious situations and for which is required the surgery revision, although the success rate does not exceed 50% of the cases; or
the use of silicone patches with occlusive action, which, although there is limited evidence of their effectiveness, they cannot be used easily in every anatomic location.

Therefore, to date is extremely felt the need of a treatment ensuring the correct healing process, without the occurrence of hypertrophic scars and preventing the formation of keloids; that has a low toxicity; that is easily administrable and with limited local side and/or systemic effects. It is also deemed that an optimal treatment, as well as effective, must be as non-invasive as possible, easy to apply and that can be performed directly by the patient without the need for a direct action of the physician.

Another type of skin lesion, besides the open wound as discussed above, is derived from treatments in which radiations are used, for example during radiotherapy sessions.

Use of radiotherapy in the treatment of cancer is based on the possibility of obtaining the total destruction of the cancer at the location of development, by avoiding as much as possible serious and irreversible alterations of the surrounding healthy tissues. The therapeutic effect is based on a selective action, which induces gradually in the cancer cells an incompatible damage with survival, but allows the normal cells to recover from the damage.

Concerning the administration technique, clinical experience shows in general the advantage of proceeding to a subdivision of the treatment in more fractions to ensure the best possible tolerance to healthy tissues at the expense of those affected by cancer. The best known fractionation scheme consists in dividing the total dose of radiations into five weekly fractions; in specific circumstances, and compatibly with the implemented technique, individual fractions can be used in high dose or very low single doses diluted over time. Very much used, especially in the past, are the split course treatments, with an interval of one or two weeks between two irradiation cycles. Despite the fundamental innovations of recent years, radiotherapy cannot yet be considered a technique completely safe and free from side effects. The radiation damages are classically divided into immediate and late. The first (similarly to chemotherapy) affect mainly the tissues with rapid multiplication, particularly susceptible to the action of ionising radiation: it is the case of the skin (dermatitis by rays, reddening of the skin, pigmentations, cracking in the irradiation field), of the mucous membranes (stomatitis, enteritis or cystitis), of the bone marrow (decrease in the number of white blood cells and platelets). The tardy reactions may appear independently from the first due to the damage sustained at the level of the connective tissue and the blood vessels: the consequences include hardening (fibrosis) of the subcutaneous tissue, inflammation of the mucous membranes comprised in the irradiation field.

Radiodermatitis is characterised by erythema, oedema and burning which regress after 3-5 days with desquamation. In the case of higher exposure, after 2-3 days from the regression of the erythematous phase, occurs a second phase with petechiae, appearance of blister droplets which then give rise to painful erosions, formation of squamous-crusts and slow repair with atropo-pigmentary, telangiectasia and skin thickened outcomes.

Acute forms can be distinguished, as being classified in grade 1 (dry) radiodermatiti, grade 2 (exudative) and grade 3 (ulcerative) and dystrophic chronic forms and necrotic ulcers.

The typical picture of the acute forms is determined in large part by the irradiation intensity: the appearance of chronic forms instead is not always in relation with the total dose of exposure, but depends on complex factors, largely on individual sensitivity, on the type of radiation and on the methods of exposure.

Often these side effects make the temporary suspension of the therapeutic scheme necessary, with the risk of a limitation of effectiveness of the radiotherapy thereof.

Therefore, to date is extremely felt also the need of a treatment ensuring the reduction in the incidence of radiodermatiti and dermatitis induced by radiations. It is deemed that an ideal treatment should provide the proper rehydration of the skin subjected to radiotherapy treatment, acting first on the correct recovery of the essential fatty acids which, at skin level, ensure maintaining in situ water molecules.

It is an object of the present invention to overcome the drawbacks previously highlighted and present in the solutions used by the state of the art, by ensuring, in case of open wounds, a correct healing process, without the occurrence of hypertrophic scars and preventing the formation of keloids, and in case of dermatitis and radiodermatiti, the proper rehydration of the skin subjected to radiotherapy treatment, by acting possibly on the correct recovery of the essential fatty acids which, at skin level, ensure maintaining in situ water molecules.

### SUMMARY OF THE INVENTION

The object indicated above has been achieved by a composition comprising virgin oil of Cannabis Sativa or a derivative thereof, and hyaluronic acid, a salt or derivative thereof.

In another aspect, the present invention relates to the use of the composition described above as a medicament, having shown to be particularly suitable for use in the treatment of skin lesions.

In particular, these skin lesions are open wounds, for which the healing process must be adjuvanted and promoted.

Alternatively, these skin lesions are seborrheic dermatitis, atopic dermatitis, irritant contact dermatitis, dermatitis herpetiformis, radiodermatitis, rashes, and similar diseases caused by radiotherapy.

The characteristics and the advantages of the present invention will be clear from the following detailed description and working examples provided for illustrative and non-limiting purposes.

### DETAILED DESCRIPTION OF THE INVENTION

Therefore, the invention relates to a composition comprising virgin oil of Cannabis sativa or a derivative thereof, and hyaluronic acid, a salt or derivative thereof.

With the expression “derivative of virgin oil of Cannabis sativa”, it is intended, for the purposes of the present invention, C₁-C₄ alkyl ester of polyunsaturated fatty acid present in the virgin oil of Cannabis sativa. Preferably, said derivative is a mixture of C₁-C₄ alkyl esters of polyunsaturated fatty acids present in the virgin oil of Cannabis sativa.

More preferably, said derivative is a mixture of ethyl esters of polyunsaturated fatty acids present in the virgin oil of Cannabis sativa.

With the expression “salt or derivative of hyaluronic acid”, it is intended, for the purposes of the present invention, sodium hyaluronate, hyaluronan ester, hyaluronan ether, hyaluronan-NHS ester, or their mixture.

In fact, it was surprisingly observed that the combination of these two components allows obtaining quickly a surprisingly good healing, at the same time avoiding the formation of hypertrophic scars and keloids, as well as reducing considerably the incidence of radiodermatiti and dermatitis induced by radiations.

The virgin oil of Cannabis sativa, preferably extracted by mechanical process of cold pressing of the peeled seeds of Cannabis sativa, has unique characteristics that make it particularly suitable for the treatment of skin diseases. In particular, in virgin oil of Cannabis sativa, it is found:
a high bioavailability of essential fatty acids in long chain (linoleic acid, α-linolenic acid, γ-linolenic acid, stearidonic acid)
a high bioavailability of vitamin E as α-tocopherol and γ-tocopherol.

The essential fatty acids play a key role in the skin barrier, by contrasting the processes of trans-epidermal junction evaporation.

In particular, linoleic acid plays the role of barrier of the cells in the stratum corneum. The construction of the permeability membrane of the stratum corneum is mainly determined by the content of these epidermal lipids. A deficiency of this essential fatty acid causes a loss of water from the epidermis making the skin dry and seborrheic. The topical application of the skin's own lipids is essential to improve the permeability of the cell membrane.

An acceleration of epidermal cell turnover, caused by chronic irritation subliminal stimuli (such as UV rays, prolonged exposure to substances modestly irritating or allergenic, etc.), inhibits cell keratinocytic differentiation with consequent alteration of the production of the lipid component. A loss of intercellular lipids is therefore observed. The water is no longer retained by the outer layers of the stratum corneum, resulting in skin xerosis.

The essential fatty acids play a normalisation function of the intercellular lipid layers, allowing the correct oxidation and degradation of the necrotic toxic organic is material that prevents the natural healing process, and ensuring the normal process of keratinisation of the skin.

Therefore, the essential fatty acids have a barrier function in the skin: in fact, they integrate the intercellular lipids present in the stratum granulosum and lower areas of the stratum corneum, by avoiding the dispersion of water and other intercellular molecules from the upper layers of the epidermis. In particular, the presence of cis-linoleic acid is essential for the arrangement of appropriate geometric lamellar layers of the epidermal lipid molecules. Lack of these essential fatty acids determines a dispersion of water from the epidermis making the skin dry.

Secondly, since every cell membrane, cytoplasmic, nuclear, mitochondrial contains essential fatty acids (incorporated as phospholipids), it is essential, in case of lesion caused by radiodermatiti, to provide a considerable recovery thereof, so as to ensure the fluidifying action of the membrane, and to allow the modulation of the activity of the protein molecules bound thereto.

The vitamin E complexes instead, applied to the skin, exert a mechanical filtering action of the UVB rays, through the double aromatic ring molecular structure that composes them. When the skin is irradiated, the ultraviolet rays cause the formation of reactive oxygen species, oxidative stress, and free radical formation.

This results in an alteration of proteins and enzyme activities, by lipid oxidation and damage to the cell membranes. The vitamin E complexes carry out a scavenger activity of free radicals and stabilise the cell membranes. A deficiency of the vitamin E complexes causes an increase of peroxides in the skin: this deficit is accentuated by the ultraviolet radiations.

The vitamin E complexes inhibit the erythema induced by ultraviolet rays and decrease the formation of sunburn cells, even if applied after irradiation. It must also be considered that the application of the vitamin E complexes causes an increase of the vitamin in the skin, an increase that persists even after irradiation.

Finally, the vitamin E complexes are able to prevent damages to collagen formation induced by the reactive oxygen species.

The particularity of the vitamin E complexes resides in that these molecules are not foreign to the human organism. Once they have been oxidised or photodecomposed, they are transformed into the normal degradation products of vitamin E of which the body knows how to get rid of, and for which no relevant toxicity is known.

However, these benefits are minor if essential fatty acids and vitamin E cannot be brought to the level of all the layers that make up the skin (epidermis, stratum corneum, and dermis), especially when this is affected by processes of lesion healing, or when this is affected by processes of constant exposure to ionising radiations (constant exposures since they are required by adherence to cancer therapy), and therefore of high stress.

By combining the virgin oil of Cannabis sativa, or derivative thereof, with hyaluronic acid (HA), a salt or derivative thereof, it was surprisingly found that it is possible to bring these active substances to the level of epidermis, the stratum corneum and the dermis.

Thanks to the high hydrophily thereof, this polysaccharide is capable of binding thereto enormous amounts of water, forming a sort of gel that fills the spaces between the collagen and the elastin fibres. This gel on one hand constitutes a water reservoir system and on the other hand functions as gelatinous matrix constituting a buffer system able to oppose mechanical stresses (bumps, deformations) to which the skin is continuously subjected.

Absence of side or unwanted effects connected to the use of hyaluronic acid is assured in that it is an element already present in the human body and in particular in an adult individual is about 15 g, predominantly localised in the skin (7.5 g).

Of fundamental importance for the applications hereby described, is the possibility of obtaining pharmaceutical compositions having rheological properties very different among each other: this depends on the intrinsic characteristics of HA, the molecules of which, before being combined with the pharmaceutical active substance, can be deeply modified through various chemical reactions.

The derivatives obtained through such reactions retain the biological characteristics of the starting polysaccharide, but have better mechanical properties; in addition, they are easily formulated in the form of hydrogels, creams, ointments, films, patches, non-woven, etc, and therefore they allow implementing a wide range of topical forms, completely adaptable to every single treatment requirement.

Preferably, the hyaluronic acid, a salt or derivative thereof, in the present invention has a molecular weight of 400 to 3,000,000 Da, more preferably of 50,000 to 1,000,000 Da.

Preferably, the composition of the invention comprises 1-30% by weight of virgin oil of Cannabis sativa or derivative thereof, and 0.05-20% by weight of hyaluronic acid, a salt or derivative thereof, on the total weight of the composition.

More preferably, the composition of the invention comprises 3-20% by weight of virgin oil of Cannabis sativa or derivative thereof, and 0.2-00% by weight of hyaluronic acid, a salt or derivative thereof, on the total weight of the composition.

As will be seen from the following Examples, these intervals have allowed to further increase the penetration ability of the skin, by maximising the release of essential fatty acids and vitamin E.

In some embodiments, in the composition of the invention, the virgin oil of Cannabis sativa or derivative thereof and the hyaluronic acid, a salt or derivative thereof, are in a weight ratio from 3:1 to 100:1.

Preferably, the virgin oil of Cannabis sativa or derivative thereof and the hyaluronic acid, a salt or derivative thereof, are in a weight ratio from 45:1 to 75:1, more preferably from 50:1 to 70:1.

The composition of the invention can comprise even cosmetically or pharmaceutically acceptable excipients.

In a first preferred embodiment, the composition of the invention consists of virgin oil of Cannabis sativa or derivative thereof, hyaluronic acid, a salt or derivative thereof, and pharmaceutically acceptable excipients.

In a second preferred embodiment, the composition of the invention consists of virgin oil of Cannabis sativa or derivative thereof, hyaluronic acid, a salt or derivative thereof, and cosmetically acceptable excipients.

In particular, suitable excipients are rheological additives, buffering agents, antimicrobial agents, antioxidant agents, antiseborrheic agents, antistatic agents, absorbent agents, UV absorbing agents, astringent agents, chelating agents, colouring agents, skin conditioning agents, preserving agents, covering agents, denaturing agents, depigmenting agents, emollients, emulsifiers, film-forming agents, gelling agents, moisturisers, hydrotropic agents, binding agents, soothing agents, smoothing agents, matting agents, skin protective agents, reducing agents, refreshing agents, sebum-restoring agents, solvents, stabilising agents, stabilising agents for emulsions, toning agents, wetting agents, or mixtures thereof.

According to a preferred embodiment, said excipients are ethyl macadamiate, tocopheryl acetate, glyceryl stearate, potassium salts of hydrolysed wheat protein palmitoyl derivatives, polyalkylacrylate, pullulan, urea, amino acids, trehalose, inositol, glucosides, hydrogenated starch, milk proteins, phenoxyethanol, methylparaben, ethylparaben, glycerin, panthenol, rethynil palmitate, ceramide, acetyl tripeptide-30, pentapeptide-18, glycoproteins, citric acid, ascorbyl palmitate, butilstearate, candelilla, ceresin, glycerol, isopropyl isostearate, isopropyl stearate, Ianolate of isopropyl, paraffin, propylene glycol, squalane, squalene, bha—butylhydroxyanisole, BHT—butylated hydroxytoluene, butyl paraoxibenzoate, bisabolol, polyacrylic acid, carrageenan, sodium dehydro acetate, dichlorophen, imidazolidinylurea, methylparaoxibenzoate, propyl paraoxibenzoate, pyrogenic silica, sorbitol, triethanolamine, or their mixtures.

In another aspect, the present invention relates to the use of the composition described above as a medicament.

In fact, as it will be seen from the following Examples, the composition has proved to be particularly suitable for use in the treatment of skin lesions.

In particular, said skin lesions are open wounds, for which the healing process must be adjuvanted and promoted.

Alternatively, said skin lesions are seborrheic dermatitis, atopic dermatitis, irritant contact dermatitis, dermatitis herpetiformis, radiodermatiti, rashes, and similar diseases caused by radiotherapy.

The composition of the invention is to be administered topically, preferably external.

Preferably, said composition is in the form of cream, emulsion, milk, ointment, patch, ointment, lotion, gel, foam or spray.

Preferably, the composition of the invention is applied to the skin to be treated in a concentration from 0.01 to 5 mg/ml, more preferably from 0.02 to 2 mg/ml.

The following are Examples of embodiment of the present invention, provided by way of illustration.

### Example 1

### Preparation of Compositions of the Invention

The following compositions were prepared, wherein the weight percentages of each component are indicated:

Composition A

| Components | % |
|---|---|
| virgin oil of Cannabis sativa | 16 |
| ethyl macadamiate | 6 |
| rethynil palmitate | 0.5 |
| glyceryl stearate, | 7 |
| potassium salts of hydrolysed wheat protein palmitoyl derivative | |
| Water | 55.6 |
| acrylates/C₁₀₋₃₀ polyalkylacrylate reticulate | 0.5 |
| hyaluronic acid | 0.25 |
| hydrolysed glycosaminoglycans | 0.05 |
| water, propylene glycol, mimosa tenuiflora extract | 5 |
| water, acetyl tripeptide-30, pentapeptide-18 | 3 |
| water, urea, aminoacids of yeast, trehalose, inositol | 2 |
| ceramide 2 | 0.2 |
| water, glycoproteins | 3 |
| phenoxyethanol, methylparaben, ethylparaben | 0.8 |
| perfume | 0.1 |

Composition B

| Components | % |
|---|---|
| oil of argania spinosa | 3 |
| virgin oil of Cannabis sativa | 13 |
| ethyl macadamiate | 3 |
| rethynil palmitate | 0.5 |
| mangifera indica butter seeds | 3 |
| glyceryl stearate, | 7 |
| potassium salts of hydrolysed wheat protein palmitoyl derivative | |
| BHT, BHA, citric acid, ascorbyl palmitate, diethylene | 0.5 |
| Water | 55.1 |
| acrylates/C₁₀₋₃₀ polyalkylacrylate reticulate | 0.5 |
| hyaluronic acid | 0.25 |
| hydrolysed glycosaminoglycans | 0.05 |
| water, propylene glycol, mimosa tenuiflora extract | 5 |
| water, acetyl tripeptide-30, pentapeptide-18 | 3 |
| water, urea, aminoacids of yeast, trehalose, inositol | 2 |
| ceramide 2 | 0.2 |
| water, glycoproteins | 3 |
| phenoxyethanol, methylparaben, ethylparaben | 0.8 |
| perfume | 0.1 |

Composition C

| Components | % |
|---|---|
| oil of argania spinosa | 3 |
| virgin oil of Cannabis sativa | 13 |
| ethyl macadamiate | 3.5 |
| mangifera indica butter seeds | 3 |
| glyceryl stearate, | 7 |
| potassium salts of hydrolysed wheat protein palmitoyl derivative | |
| BHT, BHA, citric acid, ascorbyl palmitate, diethylene | 0.5 |
| Water | 55.2 |
| acrylates/C₁₀₋₃₀ polyalkylacrylate reticulate | 0.5 |
| hyaluronic acid | 3.1 |
| hydrolysed glycosaminoglycans | 0.2 |
| water, propylene glycol, mimosa tenuiflora extract | 5 |
| water, acetyl tripeptide-30, pentapeptide-18 | 3 |
| water, urea, aminoacids of yeast, trehalose, inositol | 2.2 |
| phenoxyethanol, methylparaben, ethylparaben | 0.8 |

Composition D

| Components | % |
|---|---|
| oil of argania spinosa | 6 |
| ethylic esters of polinsaturated fat acids | 5 |
| of virgin oil of Cannabis sativa | |
| ethyl macadamiate | 5 |
| rethynil palmitate | 0.5 |
| mangifera indica butter seeds | 5 |
| glyceryl stearate, | 7 |
| potassium salts of hydrolysed wheat protein palmitoyl derivative | |
| BHT, BHA, citric acid, ascorbyl palmitate, diethylene | 0.5 |
| Water | 55.1 |
| acrylates/C₁₀₋₃₀ polyalkylacrylate reticulate | 0.5 |
| hyaluronic acid | 0.25 |
| hydrolysed glycosaminoglycans | 0.05 |
| water, propylene glycol, mimosa tenuiflora extract | 5 |
| water, acetyl tripeptide-30, pentapeptide-18 | 3 |
| water, urea, aminoacids of yeast, trehalose, inositol | 2 |
| ceramide 2 | 0.2 |
| water, glycoproteins | 3 |
| phenoxyethanol, methylparaben, ethylparaben | 0.8 |
| perfume | 0.1 |

Composition E

| Components | % |
|---|---|
| virgin oil of Cannabis sativa | 18 |
| ethyl macadamiate | 6 |
| tocopheryl acetate | 0.5 |
| glyceryl stearate, | 6.5 |
| potassium salts of hydrolysed wheat protein palmitoyl derivative | |
| Water | 57.6 |
| acrylates/C₁₀₋₃₀ polyalkylacrylate reticulate | 0.2 |
| hyaluronic acid | 0.195 |
| hydrolysed glycosaminoglycans | 0.005 |
| Pullulan | 5 |
| water, urea, aminoacids of yeast, trehalose, inositol | 1 |
| malt oligosil glucoside, amido hydrogenated | 2 |
| alcohol, water, extract of onopordum acanthium | 2 |
| milk proteins | 0.2 |
| phenoxyethanol, methylparaben, ethylparaben | 0.8 |

Composition F

| Components | % |
|---|---|
| oil of argania spinosa | 11 |
| virgin oil of Cannabis sativa | 13 |
| tocopheryl acetate | 0.5 |
| BHT, BHA, citric acid, ascorbyl palmitate, diethylene | 0.5 |
| Water | 57.6 |
| acrylates/C₁₀₋₃₀ polyalkylacrylate reticulate | 0.2 |
| hyaluronic acid | 4 |
| hydrolysed glycosaminoglycans | 0.2 |
| Glycerine | 4 |
| Pullulan | 3 |
| water, urea, aminoacids of yeast, trehalose, inositol | 1 |
| malt oligosil glucoside, amido hydrogenated | 2 |
| alcohol, water, extract of onopordum acanthium | 2 |
| milk proteins | 0.2 |
| phenoxyethanol, methylparaben, ethylparaben | 0.8 |

Composition G

| Components | % |
|---|---|
| virgin oil of Cannabis sativa | 13 |
| ethyl macadamiate | 11 |
| tocopheryl acetate | 0.5 |
| glyceryl stearate, | 6 |
| potassium salts of hydrolysed wheat protein palmitoyl derivative | |
| BHT, BHA, citric acid, ascorbyl palmitate, diethylene | 0.5 |
| Water | 57.6 |
| acrylates/C₁₀₋₃₀ polyalkylacrylate reticulate | 0.2 |
| hyaluronic ester | 0.195 |
| hydrolysed glycosaminoglycans | 0.005 |
| Glycerine | 2 |
| Panthenol | 1 |
| Pullulan | 2 |
| water, urea, aminoacids of yeast, trehalose, inositol | 1 |
| malt oligosil glucoside, amido hydrogenated | 2 |
| alcohol, water, extract of onopordum acanthium | 2.1 |
| milk proteins | 0.1 |
| phenoxyethanol, methylparaben, ethylparaben | 0.8 |

Composition H

| Components | % |
|---|---|
| oil of argania spinosa | 5 |
| virgin oil of Cannabis sativa | 13 |
| ethyl macadamiate | 6 |
| tocopheryl acetate | 0.5 |
| glyceryl stearate, | 6 |
| potassium salts of hydrolysed wheat protein palmitoyl derivative | |
| BHT, BHA, citric acid, ascorbyl palmitate, diethylene | 0.5 |
| Water | 57.6 |
| acrylates/C₁₀₋₃₀ polyalkylacrylate reticulate | 0.2 |
| hyaluronic acid | 0.195 |
| hydrolysed glycosaminoglycans | 0.005 |
| Glycerine | 2 |
| Panthenol | 1 |
| Pullulan | 2 |
| water, urea, aminoacids of yeast, trehalose, inositol | 1 |
| malt oligosil glucoside, amido hydrogenated | 2 |
| alcohol, water, extract of onopordum acanthium | 2.1 |
| milk proteins | 0.1 |
| phenoxyethanol, methylparaben, ethylparaben | 0.8 |

### Example 2

### Evaluation of Reparative/Regenerative Activity of Composition 1-B Through In Vitro Experiments on Fibroblast Cell Cultures

To evaluate the reparative/regenerative activity on fibroblasts, the in vitro analysis of the total protein synthesis was conducted.

Fibroblasts are present in the dermis, the skin layer underlying the epidermis, their task being to synthesise the collagen and the other fibres that make up the extracellular matrix of the dermis. The fibroblasts that were used in the experiments are of the primary type, derived from human dermis.

The cultured cells are treated with scalar concentrations of the tested medical device comprised between 0.03125 and 1 mg/ml.

The fibroblasts were incubated in MEM (Minimal Essential Medium)-Sodium-Pyruvate+2% fetal calf serum (FCS).

The cells, incubated with the composition 1-B for 24-48-72 hours, were isolated, centrifuged, washed in PBS, and subjected to lysis for the extraction of the total proteins. The dosage of the extracted proteins was performed by using a commercial kit (BioraD Kit). The spectrophotometer reading was performed at a wavelength of 492 nm.

The results obtained after 24 h of incubation, are given in Table 1 below.

TABLE 1

| Concentration of the sample | Total proteins | % increase |
|---|---|---|
| (mg/ml) | (μg/ml) | of proteins concentration |
| 0.5 | 38.010 | 81 |
| 0.25 | 29.932 | 42.5 |
| 0.125 | 29.939 | 42.5 |
| Control | 21 | 0 |

It is observed that the sample increased the % of total proteins with respect to the basal protein level, at all tested concentrations with a maximum (81%) at the highest tested concentration.

The results obtained after 48 h of incubation, are given in Table 2 below.

TABLE 2

| Concentration of the sample | Total proteins | % increase |
|---|---|---|
| (mg/ml) | (μg/ml) | of proteins concentration |
| 0.5 | 24.405 | 0 |
| 0.25 | 41.411 | 75.8 |
| 0.125 | 26.956 | 14.4 |
| Control | 23.554 | 0 |

It is observed that the sample increased the % of total proteins with respect to the basal protein level, for the tested concentrations comprised between 0.125 and 0.250 mg/ml with a maximum (75.8%) at the tested concentration equal to 0.250 mg/ml.

The results obtained after 72 h of incubation, are given in Table 3 below.

TABLE 3

| Concentration of the sample | Total proteins | % increase |
|---|---|---|
| (mg/ml) | (μg/ml) | of proteins concentration |
| 1* | 399.823 | 569.801 |
| 0.5 | 80.952 | 35.6 |
| 0.25 | 76.7 | 28.5 |
| 0.125 | 110.714 | 85.5 |
| Control | 59.694 | 0 |

*due to the optimum condition of the cells, a dosage of the proteins at a higher concentration was performed.

It is observed that the sample increased the % of total proteins with respect to the basal protein level, at all tested concentrations with a maximum (85.5%) at the lowest tested concentration. It was also noticed that if the cells were incubated with a dose of product equal to 1 mg/ml, the production of total proteins increased extremely.

From the above given data, it was thus shown that the composition of the invention had regenerating activity on cell cultures of human fibroblasts, suitable for use thereof as an adjuvant drug in the process of healing of open wounds.

### Example 3

### Evaluation of Reparative/Regenerative Activity of Composition 1-B Through In Vitro Experiments on Keratinocytes Cell Cultures

To evaluate the reparative/regenerative activity on keratinocytes, the in vitro analysis of the total protein synthesis was conducted

The keratinocytes that were used in the experiments are of the primary type, derived from human dermis.

The cultured cells were treated with 6 concentrations of composition 1-B: from 0.03125 to 1 mg/ml.

The fibroblasts were incubated in MEM (Minimal Essential Medium)-Sodium-Pyruvate+2% fetal calf serum (FCS).

The cells, incubated with the composition 1-B for 24 hours, were isolated, centrifuged, washed in PBS, and subjected to lysis for the extraction of the total proteins. The dosage of the extracted proteins was performed by using a commercial kit (BioraD Kit). The spectrophotometer reading was performed at a wavelength of 492 nm.

The results obtained after 24 h of incubation, are given in Table 4 below.

TABLE 4

| Concentration of the sample | Total proteins | % increase |
|---|---|---|
| (mg/ml) | (μg/ml) | of proteins concentration |
| 0.5 | 236.989 | 26.85 |
| 0.25 | 238.265 | 27.54 |
| 0.125 | 225.510 | 20.71 |
| Control | 186.819 | 0 |

It is observed that the sample increased the % of total proteins with respect to the basal protein level, at all the tested concentrations with a maximum (27.54%) at the tested concentration equal to 0.250 mg/ml.

From the above given data, it was thus shown that the composition of the invention had regenerating activity on cell cultures of human keratinocytes.

It was also carried out an MTT test for assessing the vitality of cells in vitro, as well as to identify the dose able to stimulate cell growth.

An adequate number of cells were seeded into the wells (a 96 well plate); once reached a confluence of 60-70%, it was by adding fresh medium containing scalar dilutions of the composition under examination.

The untreated cells were considered as control.

Incubation with the composition of the invention continued for 24-48-72 hours.

After replacing the medium with fresh medium supplemented with MTT, the cells were incubated for 3 hours at 37° C. Then the cells were subjected to several washings to eliminate the residues of the MTT solution. The spectrophotometer reading was performed at a wavelength of 540 nm.

The results obtained after 24 h of incubation, are given in Table 5 below.

TABLE 5

| Concentration of the sample | Total proteins |
|---|---|
| (mg/ml) | (μg/ml) |
| 1 | 15.8 |
| 0.500 | 18.5 |
| 0.250 | 15.3 |
| 0.125 | 15.9 |
| 0.0625 | 12.3 |
| 0.03125 | 7.7 |
| Control | — |

It was observed that the sample stimulated partially the cell growth at the tested concentrations comprised between 0.0625 and 1 mg/ml.

The results obtained after 48 h of incubation, are given in Table 6 below.

TABLE 6

| Concentration of the sample | Total proteins |
|---|---|
| (mg/ml) | (μg/ml) |
| 1 | 48.7 |
| 0.500 | 40.5 |
| 0.250 | 42 |
| 0.125 | 47.5 |
| 0.0625 | 47 |
| 0.03125 | 38.2 |
| Control | — |

It was observed that the sample stimulated considerably the cell growth at all the tested concentrations.

The results obtained after 72 h of incubation, are given in Table 7 below.

TABLE 7

| Concentration of the sample | Total proteins |
|---|---|
| (mg/ml) | (μg/ml) |
| 1 | 37.9 |
| 0.500 | 33.5 |
| 0.250 | 36.6 |
| 0.125 | 34 |
| 0.0625 | 37.6 |
| 0.03125 | 40.9 |
| Control | — |

It was observed that the sample stimulated considerably the cell growth at all the tested concentrations.

From the above given data, it was thus shown that the composition of the invention was able to stimulate cell growth of keratinocytes in vitro for all the tested concentrations, in particular after 48 and 72 hours of incubation; in addition, it is shown that the composition of the invention had also regenerating activity on cell cultures of human keratinocytes.

The composition of the invention was therefore suitable for use as adjuvant drug in the process of healing open wounds.

### Example 4

### Evaluation of Reparative/Regenerative Activity of Composition 1-H Through In Vitro Experiments on Fibroblast Cell Cultures

To evaluate the reparative/regenerative activity on fibroblasts, the in vitro analysis of the total protein synthesis was conducted.

The fibroblasts that were used in the experiments are of the primary type, derived from human dermis.

The cultured cells are treated with scalar concentrations of the tested medical device comprised between 0.03125 and 1 mg/ml.

The fibroblasts were incubated in MEM (Minimal Essential Medium)-Sodium-Pyruvate+2% fetal calf serum (FCS).

The cells, incubated with the composition 1-B for 24-48-72 hours, were isolated, centrifuged, washed in PBS, and subjected to lysis for the extraction of the total proteins. The dosage of the extracted proteins was performed by using a commercial kit (BioraD Kit). The spectrophotometer reading was performed at a wavelength of 492 nm.

The results obtained after 24 h of incubation, are given in Table 8 below.

TABLE 8

| Concentration of the sample | Total proteins | % increase |
|---|---|---|
| (mg/ml) | (μg/ml) | Of proteins concentration |
| 0.5 | 38.435 | 83 |
| 0.25 | 38.010 | 80.9 |
| 0.125 | 36.734 | 74.9 |
| Control | 21.003 | 0 |

It is observed that the sample increased the % of total proteins with respect to the basal protein level, at all tested concentrations with a maximum (80%) at the highest tested concentration.

The results obtained after 48 h of incubation, are given in Table 9 below.

TABLE 9

| Concentration of the sample | Total proteins | % increase |
|---|---|---|
| (mg/ml) | (μg/ml) | Of proteins concentration |
| 0.5 | 37.585 | 48.8 |
| 0.25 | 46.938 | 85.9 |
| 0.125 | 32.483 | 28.6 |
| Control | 25.255 | 0 |

It is observed that the sample increased the % of total proteins with respect to the basal protein level, for the tested concentrations comprised between 0.125 and 0.250 mg/ml with a maximum (85.9%) at the tested concentration equal to 0.250 mg/ml.

The results obtained after 72 h of incubation, are given in Table 10 below.

TABLE 10

| Concentration of the sample | Total proteins | % increase |
|---|---|---|
| (mg/ml) | (μg/ml) | Of proteins concentration |
| 1* | 391.326 | 555.5 |
| 0.5 | 85.204 | 42.7 |
| 0.25 | 76.701 | 28.5 |
| 0.125 | 68.197 | 14.2 |
| Control | 59.694 | 0 |

It is observed that the sample increased the % of total proteins with respect to the basal protein level, at all tested concentrations with a maximum (42.7%) at the lowest tested concentration. It was also noticed that if the cells were incubated with a dose of product equal to 1 mg/ml, the production of total proteins increased extremely.

From the above given data, it was thus shown that the composition of the invention had regenerating activity on cell cultures of human fibroblasts, suitable for use thereof as regenerating of the skin subjected to radiotherapy treatment.

### Example 5

### Evaluation of Reparative/Regenerative Activity of Composition 1-H Through In Vitro Experiments on Kerotinocytes Cell Cultures

To evaluate the reparative/regenerative activity on keratinocytes, the in vitro analysis of the total protein synthesis was conducted.

The keratinocytes that were used in the experiments are of the primary type, derived from human dermis.

The cultured cells are treated with 6 concentrations of the composition 1-H: from 0.03125 to 1 mg/ml.

The fibroblasts were incubated in MEM (Minimal Essential Medium)-Sodium-Pyruvate+2% fetal calf serum (FCS).

The cells, incubated with the composition 1-H for 24 hours, were isolated, centrifuged, washed in PBS, and subjected to lysis for the extraction of the total proteins. The dosage of the extracted proteins was performed by using a commercial kit (BioraD Kit). The spectrophotometer reading was performed at a wavelength of 492 nm.

The results obtained after 24 h of incubation, are given in Table 11 below.

TABLE 11

| Concentration of the sample | Total proteins | % increase |
|---|---|---|
| (mg/ml) | (μg/ml) | Of proteins concentration |
| 0.5 | 242.517 | 29.8 |
| 0.25 | 244.218 | 30.7 |
| 0.125 | 268.027 | 43.5 |
| Control | 186.819 | 0 |

It is observed that the sample increased the % of total proteins with respect to the basal protein level, at all tested concentrations with a maximum (43.5%) at the lowest tested concentration.

From the above given data, it was thus shown that the composition of the invention had regenerating activity on cell cultures of human keratinocytes.

It was also carried out an MTT test for assessing the vitality of cells in vitro, as well as to identify the dose able to stimulate cell growth.

An adequate number of cells were seeded into the wells (a 96 well plate); once reached a confluence of 60-70%, it was by adding fresh medium containing scalar dilutions of the composition under examination.

The untreated cells were considered as control.

Incubation with the composition of the invention continued for 24-48-72 hours.

After replacing the medium with fresh medium supplemented with MTT, the cells were incubated for 3 hours at 37° C. Then the cells were subjected to several washings to eliminate the residues of the MTT solution. The spectrophotometer reading was performed at a wavelength of 540 nm.

The results obtained after 24 h of incubation, are given in Table 12 below.

TABLE 12

| Concentration of the sample | Total proteins |
|---|---|
| (mg/ml) | (μg/ml) |
| 1 | 16.6 |
| 0.500 | 26.4 |
| 0.250 | 21.2 |
| 0.125 | 16.2 |
| 0.0625 | 16.3 |
| 0.03125 | 8.1 |
| Control | — |

It was observed that the sample stimulated considerably the cell growth at the tested concentrations comprised between 0.0625 and 1 mg/ml.

The results obtained after 48 h of incubation, are given in Table 13 below.

TABLE 13

| Concentration of the sample | Total proteins |
|---|---|
| (mg/ml) | (μg/ml) |
| 1 | 45.7 |
| 0.500 | 45.6 |
| 0.250 | 42.7 |
| 0.125 | 38.1 |
| 0.0625 | 37.6 |
| 0.03125 | 37.2 |
| Control | — |

It was observed that the sample stimulated considerably the cell growth at all the tested concentrations.

The results obtained after 72 h of incubation, are given in Table 14 below.

TABLE 14

| Concentration of the sample | Total proteins |
|---|---|
| (mg/ml) | (μg/ml) |
| 1 | 41.8 |
| 0.500 | 41.6 |
| 0.250 | 36.7 |
| 0.125 | 36.6 |
| 0.0625 | 34.6 |
| 0.03125 | 34.7 |
| Control | — |

It was observed that the sample stimulated considerably the cell growth at all the tested concentrations.

From the above given data, it was thus shown that the composition of the invention was able to stimulate cell growth of keratinocytes in vitro for all the tested concentrations, in particular after 48 and 72 hours of incubation; in addition, it was shown that the composition of the invention had also regenerating activity on cell cultures of human keratinocytes.

The composition of the invention was therefore suitable for use as regenerating of the skin subjected to radiotherapy treatment

From the detailed description and from the above given Examples, it was demonstrated the evident advantages achieved by the composition of the invention, which, being able to bring essential fatty acids and vitamin E at the level of all the layers that make up the skin (epidermis, the corneous layer and dermis), advantageously and surprisingly allows to support the healing processes, and to regenerate the skin subjected to ionising radiations.

1. A topical composition for treating skin lesions comprising 1-30% by weight of virgin oil of Cannabis sativa or a derivative thereof, and 0.05-20% by weight of hyaluronic acid, a salt or derivative thereof, on the total weight of the composition.

2. The composition of claim 1, wherein virgin oil of Cannabis sativa or a derivative thereof, hyaluronic acid, a salt or derivative thereof, are the only active ingredients in the treatment of skin lesions, the remaining ingredients being and pharmaceutically or cosmetically acceptable excipients.

3. The composition of claim 1, wherein hyaluronic acid, a salt or derivative thereof has a molecular weight of 400 to 3,000,000 Da.

4. The composition of claim 1, comprising 3-20% by weight of virgin oil of Cannabis sativa or a derivative thereof, and 0.2-10% by weight of hyaluronic acid, a salt or derivative thereof, on the total weight of the composition.

5. The composition of claim 1, wherein said salt or said derivative of hyaluronic acid is chosen from sodium hyaluronate, hyaluronan ester, hyaluronan amide, hyaluronan ether, hyaluronan amine, hyaluronan-NHS ester, or their mixture.

6. The composition of claim 2, wherein said pharmaceutically or cosmetically acceptable excipients are selected from the group consisting of rheological additives, buffering agents, antimicrobial agents, antioxidant agents, antiseborrheic agents, antistatic agents, absorbent agents, UV absorbing agents, astringent agents, chelating agents, colouring agents, skin conditioning agents, preserving agents, covering agents, denaturing agents, depigmenting agents, emollients, emulsifiers, film-forming agents, gelling agents, moisturizers, hydrotropic agents, binding agents, soothing agents, smoothing agents, matting agents, skin protective agents, reducing agents, refreshing agents, sebum-restoring agents, solvents, stabilizing agents, stabilizing agents for emulsions, toning agents, wetting agents, and mixtures thereof.

7. The composition of claim 1, wherein said composition is suitable for the external topical treatment of skin lesions.

8. The composition of claim 7, wherein said skin lesions are open wounds.

9. The composition of claim 7, wherein said skin lesions are seborrheic dermatitis, atopic dermatitis, irritant contact dermatitis, dermatitis herpetiformis, radiodermatitis, rashes, and similar diseases caused by radiotherapy.

10. The composition of claim 1, in the form of cream, emulsion, milk, ointment, patch, ointment, lotion, gel, foam or spray.
